(19) United States
(12) Patent Application Publication
(10) Pub. No.: US 2010/0317924 A1
(43) Pub. Date: Dec. 16, 2010

(54) DIGITAL IMAGE DATA COLLECTION APPARATUS SYSTEM AND METHOD

(76) Inventors: Michael A. Sisko, Windermere, FL (US); Glenn Ringel, Windermere, FL (US)

Correspondence Address: PATENT DEPARTMENT;LARKIN, HOFFMAN, DALY & LINDGREN, LTD., 1500 WELLS FARGO PLAZA, 7900 XERXES AVENUE SOUTH, BLOOMINGTON, MN, 55431, US

(21) Appl. No.: 12/789,578

(22) Filed: May 28, 2010

Related U.S. Application Data
(60) Provisional application No. 61/187,438, filed on Jun. 16, 2009.

(51) Int. Cl. A61B 1/227

(52) U.S. Cl. 600/200

(57) ABSTRACT

To provide additional capabilities when utilizing an otoscope apparatus, an attachment device is provided for digitally capturing medical data for ears, nose and throat patients. The device is implemented as an attachment to an otoscope, which replaces the wide angle viewing lens of the instrument head, for the purpose of digitally capturing diagnostic images and data. In practice this may typically include images of the tympanic membrane, ear canal, tharynx and nasal cavity. A method and system is utilized that assigns a unique identifier to each image, specific to each patient, based on predetermined criteria as well as the date and time. The attachment device is provided with the capability to magnify and/or reduce the image dimension via a focusable lens of the apparatus, while an internal processor displays and stores suitable images. Additionally, the device includes the ability to record notes, observations and comments via a dictation feature. The displayed and stored digital images, records, dictated notes and any related data are then categorized and retained by the system while being readily accessible for download or transmission, via a USB connection, wireless connection, network connection or portable memory card.

### RELATED APPLICATIONS

This application claims the benefit of previously filed U.S. Provisional application 61/187,438, filed Jun. 16, 2009 and entitled “Digital Image Data Collection Systems.”

### FIELD OF THE INVENTION

The present invention relates generally to an attachment for an otoscope apparatus which can be described as a portable, wireless system and method for digitally capturing medical data for ears, nose and throat patients. Naturally, this system and method is also usable to capture any data resulting from a doctor's use of an otoscope.

### BACKGROUND OF THE INVENTION

Otoscopes are hand-held medical instruments used by medical practitioners as a means to provide a visual analysis of specific areas of a patient's body, including the tympanic membrane, ear canal, tharynx and nasal cavity. In some situations, otoscopes are also used to closely examine many different areas of the body depending on the practice and habits of the medical practitioner. Existing otoscopes provide relevant light, but only one level of magnification to observe individual patient health conditions during an exam.

There are a number of perceived needs which could greatly improve the functionality of the otoscope; e.g., having a wireless device offering flexible magnification for better visualization of the tympanic membrane, “fingerprinting” the tympanic membrane, associated audio capture of the medical practitioners examination in real time, thereby establishing an auditory medical record and captured digital image of the target area. Each of these features would allow for the creation of a more complete audiovisual medical record, which would also provide an improved framework for diagnosis and/or consultation. These enhanced features should all be conveniently usable during an examination and operated with one or two fingers of one hand.

To achieve these improvements, requires an attachment or significant modification to the otoscope instrument. For example, the ability to adjust magnification to account for some patients having either long or short ear canals, as compared to a standard length ear canal would require substantial changes to the optical components of an otoscope. A device having these features would allow for accurate digital image capture of the tympanic membrane in proper focus. This proper focus combined with digital image capture would provide a distinct advantage enhancing the medical practitioner's ability to give proper diagnosis and medical care. Further, the device enables contrast enhancement methods that can be used to digitally fingerprint the tympanic membrane for measurement, treatment efficacy and other analysis.

In today's health care system there is also a continuous need to provide accurate and complete medical records. As is well understood, complete and accurate medical records provide an invaluable resource for the practitioner. Tools or systems which make this record keeping function more efficient and effective, while also expanding record keeping capabilities, are valuable and welcome additions to the medical practitioners resources.

In summary, a need exists for an otoscope device attachment, system and method for enhancing the audiovisual features and capability of a medical practitioner, capturing detailed dictation and accurate digital images, and for storing an audiovisual record at the time of examination.

### SUMMARY OF THE INVENTION

The present invention relates to a device and apparatus that provides additional features and tools to a medical practitioner. One embodiment of the invention is an attachment to an otoscope, replacing the static, one view, wide angle viewing lens of the instrument head, for the purpose of providing flexible magnification and digitally capturing diagnostic images and data. In practice, this data will typically include images of the tympanic membrane, ear canal, tharynx and nasal cavity, and other relevant examination points. A preferred method and system is utilized that assigns a unique identifier to each image or audio recording, specific to each patient, based on predetermined criteria and automatically stores all captured data with this identifier, thus memorializing the examination date and time.

The attachment device also provides the capability to magnify and reduce the image dimension via a focusable lens of the apparatus enabling multiple images of the examination target. An internal processor then displays the images for medical practitioner selection or deletion and then conveniently stores the most suitable, selected images. The digital images are also categorized and retained by the system while being readily accessible for download or transmission, by authorized personnel for diagnosis or consultation. The transfer of these images can be carried out via a USB port, wireless transmitter/receiver or portable memory card. Additionally, the device is capable of producing corresponding observation via sound recordings (e.g. dictation), which are then coupled with the above identified visual data to provide a more complete record of the examination.

For the reasons outlined above, it is an object of the present invention to provide an attachment to an otoscope apparatus having the capabilities to remedy the above-noted deficiencies in the prior art.

It is one further goal of the present invention to provide an attachment device with communication capabilities for transmitting captured data (e.g. images, sound recordings, etc.) or for receiving corresponding patient information (e.g. patient file numbers, diagnostic tools, etc.). This may include a wireless device that can be conveniently attached to existing otoscope designs thus providing increased functionality. This may also include a device with network connection capabilities (e.g. via USB connections, RS232 connections, IEEE 1394 connections, etc.). These features provide simplified audiovisual logistics management and data transfer for medical record keeping.

When combined with the otoscope, the attachment device provides a greater flexible field of view with selective suitable magnification, or focusing. Preferred embodiments also include anti-shake capability and an image time delay feature enabling the medical practitioner the necessary time to confirm image quality.

Features of the present otoscope attachment reduces the amount of time required for focusing on the target area, capturing the digital image and establishing a permanent visual medical record. Further, the user is easily able to review and delete images on the wireless device, yielding selectivity at the time of examination for storing the most suitable diagnostic images. The LCD screen facilitates the review of the tympanic membrane for ear infections, external otitis, otitis media, serous otitis media, perforation, i.e. abnormal findings in the least amount of time. The device endeavors to provide the platform to carry out and record a quick, one click per target area examination.

By providing several of the above-referenced features as an attachment, unique functionality and flexibility is provided. This attachment to an otoscope produces an optical system that provides a real time dictation capability, a focusable digital imager with enhanced color clarity and anti-shake functionality, thereby achieving a digital audiovisual otoscope.

In addition to the various features discussed above, the audio interface can be easily utilized to memorialize or at the examiners discretion to dictate information specifying patient specific examination information, such as identifying image target, e.g. right ear, left ear, etc., patient age, specific patient ailment, and state the physical findings and diagnosis. This can then be used by the medical practitioner, along with other settings and recording features, to digitally capture images and information most relevant for the particular patient involved. Specific operational characteristics and settings are easily creatable through a convenient interface system using particular keys or buttons. Depending on the physical characteristics of the patient and the corresponding target area to be digitally captured, recording of useful diagnostic information is thus conveniently achieved.

By using a flexible drop down menu interface, accessible using keys or buttons which are designed for one hand operation, the medical practitioner can also achieve patient specific encoding which allows the captured digital images to be cross referenced with the specific patient. This may include the correlation of audio recordings with other image or data files, or may include behind the scenes file storage techniques used by the operating system, using data input from the user. This allows for accurate real time record keeping while automatically archiving the patient identification, time and date of each image captured. This interface is intended to shorten the examination. This is particularly useful with younger patients. Children are often traumatized by the medical practitioner invading their personal space and this device shortens the timeframe for the examiner to capture an image, view and diagnose the observation.

One advantageous feature of the present invention is that the medical practitioner can use the instrument as an attachment to an existing otoscope without the need for additional training as the interface is thoughtfully designed to be both simple and intuitive.

Another advantageous feature of the present invention is that a medical practitioner can preset the device according to a given patient specific physiological structure, reducing the time needed to effectively use the device. With this capability of pre-setting the device for age (e.g. adult or child), generally, or individual patient specifically, the practitioner can easily and more quickly capture the image, capture the examination with audio dictation and establish a diagnosis while simultaneously creating a comprehensive digital record.

An additional advantageous feature achieved by the attachment device is its configuration which allows for one-handed operation. The shutter button, audio record button and other controls are well proportioned dimensionally and thoughtfully located on the device, enabling easy execution even if the patient is an upset child. The size of the LCD screen provides a quality image for evaluation and review with the patient, or patient's guardian. Displaying the image during the examination aids in patient/guardian education, improves diagnosis and increases patient/guardian compliance with the prescribed treatment.

A further advantageous feature achieved by the device is its contrast enhancement capabilities and related methods that may be used to digitally fingerprint the tympanic membrane for measurement, treatment options, treatment efficacy and other analysis. When examining the tympanic membrane, the chorda tympani nerve and arteries pass through the layers of the superior portion of the membrane and the ridges may be captured by the device, much like a fingerprint. The device of the present invention is able to produce a digital record of the vasculature, nerves, cartilage and bones within the depth of field associated with the membrane. This information could then be used for multiple purposes.

These features and advantages are apparent from the following detailed description and related drawings.

### BRIEF DESCRIPTION OF THE DRAWINGS

FIGS. 1-4 are various views of the device as attached to an otoscope.

FIG. 5 is a perspective view of the attachment alone.

FIG. 6 is a flow chart illustrating one example of the data flow used to accurately store all collected data.

FIGS. 7-8 are perspective views showing right hand and left hand use.

### DETAILED DESCRIPTION OF PREFERRED EMBODIMENTS

As mentioned above, the present invention generally provides the capability for medical practitioners to easily dictate observations, capture, save and transfer, both images and examination information while utilizing a wireless otoscope. Outlined below is a more detailed description of various embodiments utilized to carry out this particular function.

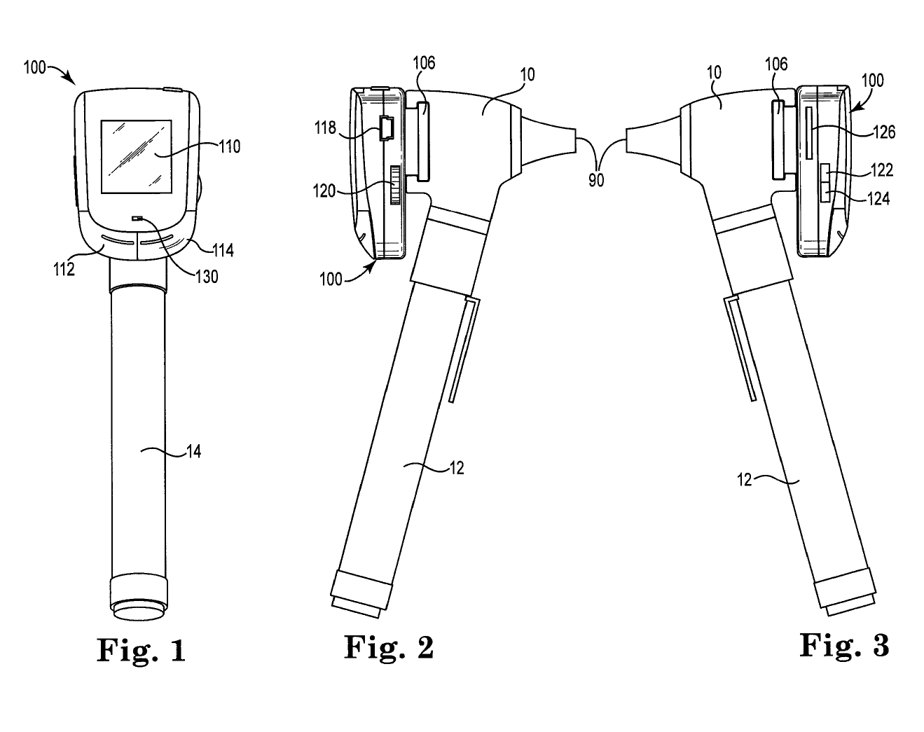

Generally illustrated in FIGS. 1-4 is an otoscope device 10 including an attachment 100 capable of displaying images present at the otoscope speculum 90 and appropriately capturing those images for later use. Further, the device provides the ability to attach or append related information, including dictation, patient identifiers, notes or related diagnostic information. As well understood, Otoscope device 10 includes a handle portion 12 and an optical head 14. Handle portion 12 typically carries batteries to provide power as necessary. Optical head 14 further includes a light or lump to illuminate the desired area, and appropriate openings or optics to allow viewing by the medical practitioner (not shown in FIGS. 1-4).

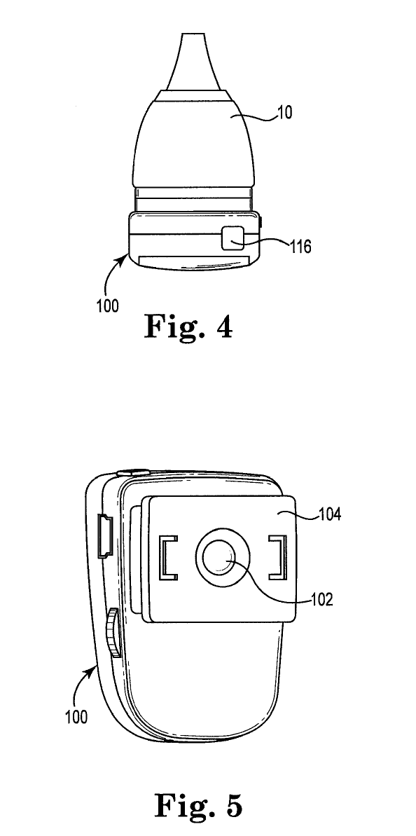

FIG. 5 illustrates a perspective view of the data collection device 100, when unattached to an otoscope. As illustrated, data collection device 100 includes a camera lens 102 which is centrally located on the back side thereof. As suggested above, data collection device 100 is configured for attachment to otoscope 10, as illustrated in FIGS. 1-4. As best illustrated in FIGS. 2 and 3, otoscope 10 and data collection device 100 each include coupling structures, thus allowing data collection device 100 to be removably attachable to otoscope 10. In this particular embodiment, data collection device 100 includes an extension 104 having attachment tabs 106 which are configured to mate with corresponding structures on otoscope 10. More specifically, a back side of otoscope 10 will include a slot appropriately configured to receive tab 106. When attached, camera lens 102 will be appropriately aligned with the optical opening contained within optical head 14 of otoscope 10. In this manner, camera lens 102, and related image capture circuitry provided inside of data collection device 100 are able to capture images of scenes provided through otoscope 10. In practice, this will likely include close examination of ear cavities, nasal cavities, the throat and related mouth structures, along with any other examination procedures conducted by a practitioner.

Referring again to FIG. 1, data collection device 100 further includes a display screen 110 positioned on a back side of data collection device 100. Generally speaking, display screen 110 is an LCD screen capable of displaying images captured through camera lens 102. In addition to display screen 110, data collection device also includes a record button 112 and a photo button 114 both positioned adjacent to display 110. Both of these buttons are ideally positioned to allow easy access by a user when the otoscope device 10 is held within their hand. Centrally located is a microphone 130, for convenient access when recording dictation of the examination observations.

Referring to FIG. 4, a top view of otoscope 10 and data collection device 100 is shown. On a top surface of data collection device 100 is a power button 116 which is utilized to turn the device on and off. The bottom of the device (not shown) will also include an AC power port for recharging the device. It is anticipated that the device is battery powered using replaceable batteries or rechargeable batteries. The AC power port is provided with the device to facilitate recharging.

Referring now to FIGS. 2 and 3, opposite side views of otoscope 10 and data collection device 100 are illustrated. On one side of the device (illustrated in FIG. 2) are located a data bus connection 118 and a zoom knob 120. Data bus connection 118 may include a USB type connection, or may also include other types of network connections. For example, an IEEE-1394 connection or an RS232 connection could also be utilized. Although the present embodiment illustrates a data bus connection, it is also understood that the device could equally utilize wireless capabilities, including Bluetooth capability or wireless Ethernet connections to achieve connectability with other systems. The data collection device 100 improves medical records through image sharing, both with the patient and/or their guardian at the time of capture, thereby improving patient/guarding education. In addition, this leads to more accurate diagnosis and patient/guardian compliance with prescribed treatment. Further, sharing the audiovisual record created by the device aids in diagnosis should a second opinion be desired, or shared with another physician. Additionally, the audiovisual record created by the device establishes an initial baseline that can be referred to at a subsequent date to evaluate the efficacy of the prescribed treatment.

On an opposite side of data collection device 100, additional control buttons are provided. More specifically, a “select” button 122 and a “delete” button 124 are positioned on a lower portion of this side. In addition, a memory card slot 126 is provided. Both select button 122 and delete button 124 will generally be utilized to carry out certain functions as provided on internal menus. As would be anticipated, these buttons allow for selections of certain modes, deletion of undesired data, etc. Memory card slot 126 is relatively self-explanatory, and includes an opening for insertion of a memory card. Naturally, internal circuitry will allow for an interface to this memory card, thereby allowing images to be stored thereon if desired.

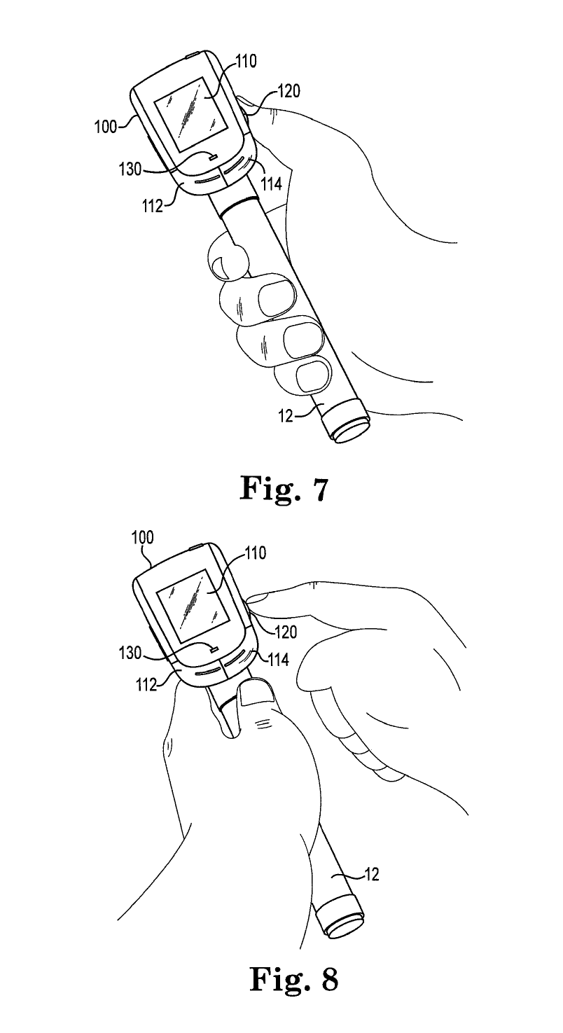

Referring now to FIGS. 7 and 8, two “application” drawings are illustrated. More specifically, these two drawings illustrate perspective views of the otoscope 10 and data collection device 100 being utilized. FIG. 7 illustrates the system being operated by a right hand while FIG. 8 illustrates the system being operated by a left hand.

As generally suggested above, the data collection device 100 includes many different features and capabilities, all contained within one single device. One primary feature is the display and image capture capabilities provided. More specifically, the internal circuitry provided within data collection device allows for images to be displayed upon LCD screen 110 and captured as desired (i.e. stored within internal memory). Utilizing the network connection or data bus connection 118, these images can easily be retrieved and/or transferred to related computer systems as necessary.

In addition to the straightforward image capture capabilities, the data collection device 100 also includes a dictation and voice capture feature, thus allowing the practitioner to record notes, comments and other observations while an examination is ongoing. To further enhance the examination and medical record detail, the audio recording feature allows the medical practitioner to explain the observations in real time, in their own words, thereby establishing and linking both visual and auditory medical records of the examination digitally. Naturally, these notes and/or observations could be attached to or coupled with a particular captured image, thus creating one data file providing valuable information related to a particular patient. It is further anticipated that a menu driven operating system and related input capabilities are provided to allow further storage of additional related information. More specifically, this would typically include patient identifiers, patient names, file numbers, etc. which could also thereby be associated with an overall digital file captured by the device. Specifically, the overall file may then include identifiers and/or necessary patient information, recorded voice data, and captured image data. The file storage system contained within the device is thus capable of correlating or associating all of this various information, thereby providing data storage consistency. It is then anticipated that this correlated information is then easily transferable to a patient record system capable of more comprehensive medical records management.

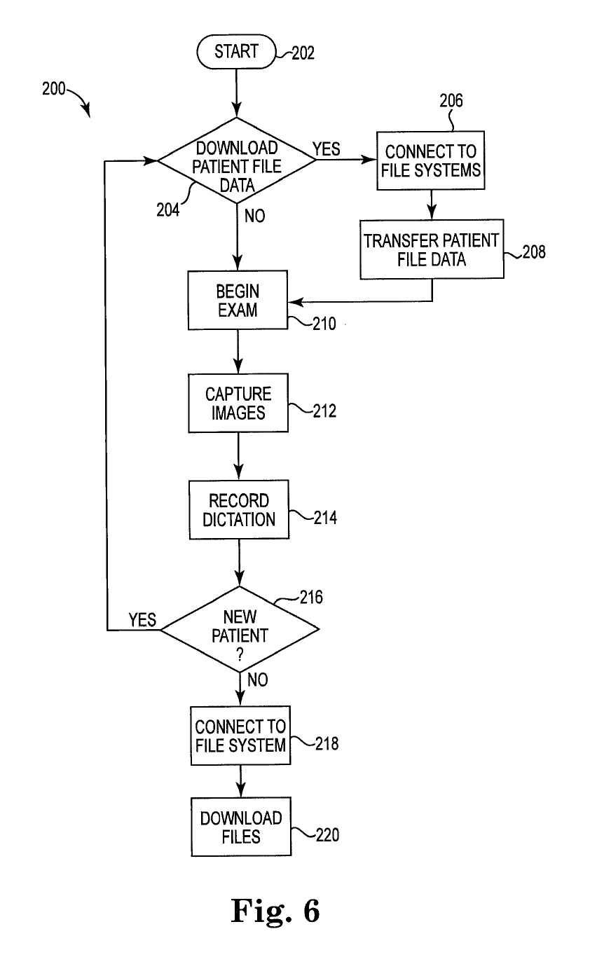

To generally outline the one example of the anticipated use, FIG. 6 provides a general flowchart showing one example of the anticipated dataflow process 200. As illustrated, process 200 starts at a starting point 202 and begins by asking whether patient file data should be downloaded to the device at step 204. As an example, potential patient file data may simply include the patient's name, internal file numbers or record keeping codes, and any other general information regarding the patient (e.g., age). If such information is desired, the process moves to step 206 where the device is connected to a file system. This would most likely be a desktop computer possibly located in the examination room, or at a nurse station. Once connected, the patient file data is then appropriately transferred to the handheld device in step 208. The process then moves to step 210 where the examination is begun. Naturally, if the patient file data is not necessary, the transfer steps can be skipped, and the practitioner could move directly to the examination stage. As discussed in detail above, the practitioner will capture images during the examination (generally shown in step 212) and will likely record dictation (generally shown in step 214). Naturally, this could take on many forms and could be relatively straight forward involving a single image and a single comment, or may involve the capturing of several images. Each of the manipulation and storage steps generally outlined above are utilized in this process.

At this point, records for the particular patient examination data will be captured in the device and the practitioner could move on to a new patient. If a new patient is desired, the process moves back to step 204 to essentially restart the process with regard to a new patient. It is anticipated that the device of the present invention will include the necessary storage capability to record information related to several patients if desired. Eventually, a file transfer process will be necessary however, which is shown at steps 218 & 220. Specifically, the process then requires the device to be connected to the file systems at step 218. Once connected, the audiovisual data is downloaded at step 220.

To provide similar capabilities, the data storage slot 126 could be utilized to store the various types of information to a removable data storage device. Subsequently, this information could be transferred as desired or necessary, using the removable data storage device (e.g. SD memory card, mini-SD card, etc).

Although generally discussed above, the image processing capabilities provided by the electronics of data capture device 100 also provides additional features and benefits. More specifically, these features allow the practitioner to zoom, resize and otherwise manipulate images as they are being captured. In this manner, the practitioner is provided with a digital zoom capability and consequently a very valuable tool for examination and diagnosis of patient conditions.

As mentioned above, data storage device 100 is removably attached to an otoscope 10 for appropriate use. By being selectively attachable, the data storage device could easily be transferred from one otoscope to another. Obviously this allows interchangeability and flexibility for the practitioner. This flexibility also allows health care systems and clinics to more easily manage their capital expenditures.

Those skilled in the art will further appreciate that the present invention may be embodied in other specific forms without departing from the spirit or central attributes thereof.

In that the foregoing description of the present invention discloses only exemplary embodiments thereof, it is to be understood that other variations are contemplated as being within the scope of the present invention. Accordingly, the present invention is not limited in the particular embodiments which have been described in detail therein. Rather, reference should be made to the appended claims as indicative of the scope and content of the present invention.

What is claimed is:

1. A portable data collection device for capturing diagnostic digital images attachable to an otoscope, utilizing the otoscope's speculum and light source for illuminating a desired object to be digitally captured, the data collection device comprising:
a lens for providing optical images of a view through the otoscope;
a digital image capture device for receiving and capturing the optical images provided by the lens;
a digital audio capture device for receiving and capturing audio signals;
a processor for receiving and temporarily storing the captured digital images and audio signals in a data file;
a memory for archiving the data file;
a display screen for instant viewing temporarily stored digital images by the processor;
a user interface comprising buttons to digitally encode and identify the data file in a manner to be unique to a given patient; and
a communications platform for transferring the data file to a medical practitioner patient records computer system.

2. The portable data collection device of claim 1 wherein the communication platform further includes USB port and portable memory.

3. The portable data collection device of claim 1 wherein the processor is capable of further refining the captured images, including increasing and decreasing the magnification of an image target.

4. The portable data collection device of claim 1 wherein the user interface further comprises a thumb wheel to enhance operation of the device.

5. The portable data collection device of claim 1 wherein the user interface includes a menu driven program further contained within the memory to accommodate the operation of the image capture device, wherein predetermined menu screens are displayed which provide certain functions to the buttons causing predetermined acts to occur.

6. The portable data collection device of claim 1 wherein the display screen is an LCD display.

7. A data collection device for use when attached to a medical device, the data collection device comprising:
a mounting bracket to accommodate attachment to the medical device;
an image collection lens;
an image sensor aligned with the image collection lens to detect an image projected through the image collection lens;
an audio sensor to capture an audio signal;
a memory for storing the image detected by the image sensor and the audio signal;
a display for presenting the detected image to a user;
at least one interface control to allow the user to manipulate the detected image and to cause the detected image to be stored in the memory; and
a control causing the audio signal to be stored in the memory and affiliated with the detected image.

8. The data collection device of claim 7 wherein the interface control includes a plurality of interface buttons positioned adjacent the display screen.

9. The data collection device of claim 7 wherein the display is an LCD display screen.

10. The data collection device of claim 7 wherein the display is a touchscreen display.

11. The data collection device of claim 7 wherein the memory contains computer executable code to implement a menu driven program to allow the user to selectively store the detected image.

12. The data collection device of claim 7 wherein the audio sensor comprises a microphone and voice capture circuitry to detect and capture audio signals.

13. The data collection device of claim 7 further comprising image manipulation software stored in the memory to allow the user to manipulate the detected image.

14. The data collection device of claim 13 wherein the image manipulation software allows the user to zoom in on selected portions of the detected image.

15. The data collection device of claim 7 further comprising a networking device to allow the user to transfer the detected image to a subsequent system.

16. The data collection device of claim 15 wherein the networking device is a wireless transmitter.

17. The data collection device of claim 15 wherein the networking device is a network connector.

18. The data collection device of claim 7 further comprising a memory slot capable of receiving removable memory device.

19. A data collection device attachable to an otoscope for capturing and storing patient data obtained during a patient examination, the data collection device comprising:
a housing having a mounting structure configured to connect to an otoscope;
a optical lens positioned within the housing and aligned with the mounting structure so that the optical lens is aligned to receive any image viewable through the otoscope;
an image sensor contained within the housing and aligned with the optical lens so that images viewable through the otoscope can be digitally captured;
a microphone for receiving audio signals;
a processor coupled to the image sensor and the microphone for temporarily storing the captured image and providing the capability to manipulate the image, the processor for further storing and processing the audio signals received by the microphone;
a memory coupled to the processor for selectively storing the captured images and the captured audio signals;
a display contained within the housing and coupled to the processor for displaying the captured images and a plurality of operational menus; and;
a user interface comprising a plurality of buttons, wherein the memory contains a menu driven program operable using the user interface to allow a user to selectively capture the images, capture related audio signals, manipulate images, and store selected data in the memory.
